(19) United States
(12) Patent Application Publication
(10) Pub. No.: US 2024/0033246 A1
(43) Pub. Date: Feb. 1, 2024

(54) COMPOUNDS FOR TREATING EYE DISEASES AND DISORDERS

(71) Applicants: The State of Israel, Ministry of Agriculture & Rural Development, Agricultural Research Organiza, Rishon Lezion (IL); Tel Hashomer Medical Research Infrastructure and Services Ltd., Tel Hashome (IL)

(72) Inventors: Anat Elmann, Rehovot (IL); Ygal Rotenstreich, Kfar-Bilu (IL); Ifat Sher, Shoham (IL)

(21) Appl. No.: 18/256,747

(22) PCT Filed: Dec. 9, 2021
(86) PCT No.: PCT/IL2021/051470

§ 371 Date: Jun. 9, 2023

Related U.S. Application Data
(60) Provisional application No. 63/123,835, filed on Dec. 10, 2020.

(51) Int. Cl. A61K 31/353; A61P 27/02; A61K 9/00; A61K 36/28

(52) U.S. Cl. CPC A61K 31/353; A61P 27/02; A61K 9/0048; A61K 36/28

(57) ABSTRACT

The invention concerns 3,5,4′-trihydroxy-6,7,3′-trimethoxyflavone (TTF) or a pharmaceutically acceptable salt or solvate thereof for use in treating, preventing, or ameliorating an eye disease or disorder, or for providing adjunct treatment to an ocular therapeutic procedure. The invention also concerns ophthalmic compositions comprising TTF.

### TECHNOLOGICAL FIELD

The present disclosure generally relates to a novel treatment of eye diseases and disorders.

### BACKGROUND ART

References considered to be relevant as background to the presently disclosed subject matter are listed below:
[1] Wong, W.L., Su, X., Li, X., Cheung, C.M.G., Klein, R., Cheng, C.-Y., and Wong, T.Y. (2014). Global prevalence of age-related macular degeneration and disease burden projection for 2020 and 2040: a systematic review and meta-analysis. Lancet Glob. Health 2, e106-16.
[2] Sunness, J.S. (1999). The natural history of geographic atrophy, the advanced atrophic form of age-related macular degeneration. Mol. Vis. 5, 25.
[3] Bhutto, I., and Lutty, G. (2012). Understanding age-related macular degeneration (AMD): relationships between the photoreceptor/retinal pigment epithelium/Bruch's membrane/choriocapillaris complex. Mol. Aspects Med. 33, 295-317.
[4] Hartong, D. T., Berson, E. L., and Dryja, T. P. (2006). Retinitis pigmentosa. Lancet 368, 1795-1809.
[5] Bravo-Gil, N., Gonzalez-Del Pozo, M., Martin-Sinchez, M., M6ndez-Vidal, C., Rodriguez-de la Rna, E., Borrego, S., and Anti5olo, G. (2017). Unravelling the genetic basis of simplex Retinitis Pigmentosa cases. Sci. Rep. 7, 41937.
[6] Patel, N., Aldahmesh, M. A., Alkuraya, H., Anazi, S., Alsharif, H., Khan, A. O., Sunker, A., Al-Mohsen, S., Abboud, E. B., Nowilaty, S. R., et al. (2016). Expanding the clinical, allelic, and locus heterogeneity of retinal dystrophies. Genet. Med. 18, 554-562.

Acknowledgement of the above references herein is not to be inferred as meaning that these are in any way relevant to the patentability of the presently disclosed subject matter.

### BACKGROUND

Age-Related Macular Degeneration (AMD) is the leading cause of blindness and severe visual impairment in the industrial world.

The prevalence of AMD is predicted to increase due to the exponential population ageing, reaching 288 million in 2040.[1] It is a well-established disease of aging, chronic oxidative stress and inflammation that ultimately lead to protein damage, aggregation, and degeneration of retinal epithelial cells (RPE) and concomitant loss of photoreceptor cells.

The hallmark of the disease condition is the presence of extracellular deposits called drusen, yellow deposits of lipids and proteins, primarily in the area of the central region of retina called macula which is the part of the retina responsible for our visual acuity.

Accumulation of drusen between the RPE and their underlying basement membrane accelerates RPE cell death that leads to photoreceptor degeneration. AMD progression and severity are directly correlated to the number and size of drusen.

Advanced AMD occurs in 2 forms: (i) Dry AMD including geographic atrophy (GA) of the RPE and overlying photoreceptors, Drusen, and (ii) choroidal neovascularization (CNV, also called “wet” AMD). Dry (GA) AMD is characterized by confluent areas of photoreceptor and RPE cell death and is responsible for 10% of the legal blindness caused by AMD.[2]

Approximately 1 million people in the USA are currently affected by Dry AMD with more than half of the patients occurring bilaterally. “Wet” (neovascular) AMD accounts for the remaining 90% of acute blindness caused by AMD and is characterized by abnormal blood vessel growth under the macula. These new vessels are largely malformed, which leads to the improper vascular integrity causing undesirable fluid leakage within the disrupted tissue infiltrated by the unwanted vasculature.[3]

Although there are treatments for “wet” AMD, which decrease neovascularization and improve vision, photoreceptors cell death continues to progress and there is no effective treatment that slows down or stops the degeneration of the retinal neurons. Furthermore, despite the prevalence of this disease, its etiology remains largely unknown.

Retinitis pigmentosa (RP) is a complex group of incurable hereditary retinal dystrophies that is characterized by progressive degeneration of rod and cone photoreceptors.

The worldwide prevalence of RP is approximately one in 3500 with a total of more than 1.5 million affected individuals.[4]

The disease can be inherited as an autosomal-recessive (about 50-60% of cases), autosomal dominant (30-40%) or X-linked (5-15%) trait. Eighty-one known causative genes and thousands of mutations have been identified so far.[5]

The first clinical presentation of RP is night blindness, often starting in adolescence, followed by progressive loss of peripheral vision and in many cases ending in loss of central vision and complete blindness in midlife. These visual symptoms reflect the gradual degeneration of rods, which mediate achromatic night vision, followed by loss of cones, which are critical for high acuity central vision.

Currently there is no approved treatment for RP, except a single gene therapy treatment that was recently approved by the FDA for patients with mutations in RPE65 gene, that are found in ˜7% of RP patients.[6] Notably the extremely high cost of this treatment (˜950,000 USD per patient) suggests it will be not accessible to majority of these patients.

Currently there is no treatment for retinal degeneration and other retinal cell damage such as in AMD, RP, and diabetic retinopathy affecting millions of patients worldwide. Recently gene therapy was approved for one of the 80 causative genes of RP, but it is beneficial for few RP patients who carry mutations in that gene. These diseases are highly heterogeneous with dozens of known causative genes. Many patients cannot be genetically diagnosed, and disease progression varies between individuals regardless of the affected gene. Hence, gene therapy will not be efficient and reasonable for all patients. Hence there is an urgent need to develop a treatment that can slow down or stop degeneration of retinal neurons to prevent blindness and to reduce the vast social and economic burden of these blinding diseases.

WO 2015/079390 discloses 3,5,4′-trihydroxy-6,7,3′-trimethoxyflavone and achillolid A isolated from Achillea Fragrantissima and showed the in vitro effects of these compounds on astrocytes, neuronal cells, and microglia suggesting their use in treatment of Alzheimer's disease, Parkinson's disease and additional brain associated neurodegenerative diseases.

### GENERAL DESCRIPTION

In a first of its aspects, the present invention provides 3,5,4′-trihydroxy-6,7,3′-trimethoxyflavone (TTF) or a pharmaceutically acceptable salt or solvate thereof for use in treating, preventing, or ameliorating an eye disease or disorder, or for providing adjunct treatment to an ocular therapeutic procedure.

In another aspect, the present invention provides TTF or a pharmaceutically acceptable salt or solvate thereof for use in a method of treating, preventing, or ameliorating eye disease or disorder, or for providing adjunct treatment to an ocular therapeutic procedure in a subject in need thereof, the method comprising administering to the subject a therapeutically effective amount of said TTF or salt or solvate thereof.

In other aspects, the present invention provides TTF or a pharmaceutically acceptable salt or solvate thereof for use in inhibition or reduction of retinal immune cells activation, in inhibition or reduction of photoreceptors death, or in prevention of retinal cell degeneration.

In another aspect, the present invention provides an ophthalmic composition comprising TTF or a pharmaceutically acceptable salt or solvate thereof, and an ophthalmic acceptable carrier.

In an embodiment the ophthalmic composition according to the invention is for use in a method of treating, preventing, or ameliorating an eye disease or disorder, or for providing adjunct treatment to an ocular therapeutic procedure in a subject, wherein said eye disease or disorder is selected from the group consisting of retinitis pigmentosa (RP), Diabetic retinopathy (DR), chorioretinitis, choroiditis, retinitis, retinochoroiditis, solar retinopathy, choroidal degeneration, choroideremia, hypertensive retinopathy, retinopathy, retinopathy of prematurity, age-related macular degeneration (AMD), macular degeneration, bull's eye maculopathy, epiretinal membrane, peripheral retinal degradation, hereditary retinal dystrophy, retinal haemorrhage, central serous retinopathy, glaucoma, optic neuropathy, leber's hereditary optic neuropathy, optic disc drusen, skleritis, keratitis, corneal ulcer, arc eye, thygeson's superficial punctate keratopathy, corneal neovascularization, corneal dystrophy, fuchs' dystrophy, keratoconus, keratoconjunctivitis sicca, herpes, dry eye, iritis, uveitis, optic neuritis, bacterial infections (e.g. Lyme disease), viral infections (e.g. measles, mumps), sarcoidosis, lupus neuromyelitis optica, eye complications associated with use of medications (e.g., quinine, antibiotics), optic nerve degeneration, ischemic optic neuropathy (e.g., Non-Arteritic Anterior Ischemic Optic Neuropathy (NAION), Anterior Ischemic Optic Neuropathy (AION), Posterior Ischemic Optic Neuropathy (PAION)).

In another aspect, the present invention provides a method of treating an eye disease or disorder, or a method for providing adjunct treatment to an ocular therapeutic procedure the method comprising administering to a subject in need thereof a therapeutically effective amount of TTF or a pharmaceutically acceptable salt or solvate thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents the number of M-cone opsin positively stained cells/mm retina obtained from RPE65/rd12 mice. The retina was incubated in media supplemented with 8 nM natural TTF (nTTF), 8 nM synthetic TTF (sTTF) or vehicle (control). Data are presented as mean±SE. pₐ represents the statistical significance of the difference between nTTF or sTTF and the control; P_{b} represents the statistical significance of the difference between nTTF and sTTF.

FIG. 2 presents TTF concentration (μg/ml) (and also presented in nM) in anterior chamber tap (eye fluids) removed at indicated time points (45 min or 3 hr) after TTF application to the eye, as determined by HPLC analysis.

FIG. 3A-3L shows representative RPE in retina flat mounts of RPE65/rd12 mice and wild type C57BL mice stained for the microglial specific marker Iba-1. A-C and G-I —show a 3 mm² area around the visual nerve entrance (x4). D-F & J-L show a larger magnification (x10).

FIG. 3M is a graph showing quantification of infiltrated microglia. A two tailed t-test was preformed (P<0.001), for both groups, i.e., the RPE65/rd12 mice and wild type C57BL mice.

FIG. 4 is a graph showing quantification of infiltrating subretinal microglia in RPE65/rd12 mice treated in vivo with eye drops containing placebo (DMSO, n=9) or TTF (n=10). The retinas were stained with the microglial specific marker Iba-1. The p value for two tailed t-test for the placebo and TTF treated groups is presented.

FIG. 5 presents IL-1 beta concentration (μg/mg retinal protein) in retinal lysates obtained from RPE65/rd12 mice treated with the vehicle (DMSO) or TTF eye drops. Lower levels of IL-1beta were demonstrated in the TTF-treated retina.

FIG. 6 presents the number of TUNEL positive cells in the photoreceptor cell layer as an indication for photoreceptor cell apoptosis. The graph presents results for a control group (RPE65/rd12 mice treated with the vehicle DMSO), versus a group of RPE65/rd12 mice treated with TTF eye drops.

FIG. 7 presents an assessment of retinal function measured by electroretinogram testing and recording of maximal dark-adapted ERG a-wave and b-wave amplitudes (pV). Two groups of RPE65/rd12 mice were assessed, where one was treated with TTF eye drops in DMSO and the other with DMSO alone (control).

### DETAILED DESCRIPTION OF EMBODIMENTS

The present invention is based on the surprising finding that 3,5,4′-trihydroxy-6,7,3′-trimethoxyflavone (TTF) protects retinal photoreceptors from cell death both in vitro and in vivo and preserves retinal function in a mouse model of Retinitis pigmentosa.

Thus, in a first aspect, the present invention provides 3,5,4′-trihydroxy-6,7,3′-trimethoxyflavone (TTF), or a pharmaceutically acceptable salt or solvate thereof, for treating, preventing, or ameliorating an eye disease or disorder in a subject, or for providing adjunct treatment to an ocular therapeutic procedure.

In another aspect, the present invention provides TTF or a pharmaceutically acceptable salt or solvate thereof for use in a method of treating, preventing, or ameliorating eye disease or disorder in a subject in need thereof, the method comprising administering to the subject a therapeutically effective amount of said TTF or a pharmaceutically acceptable salt or solvate thereof.

The structure of the TTF compound appears in formula VIII:

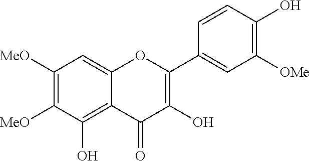

wherein Me represents a methyl group.

TTF may be isolated from a plant, e.g., Achilleafragrantissima (Af) which is a desert plant used in traditional medicine, as described for example in WO 2015/079390. Such isolated TTF is also referred to herein as “natural TTF”.

The following is an exemplary procedure for isolation of natural TTF: Achillea fragrantissima may be collected in various desert regions. As a non-limiting example, it may be collected in the Arava Valley, Israel.

Sun or air or oven or freeze-dried Af (1 kg) is homogenized and extracted with petrol ether (3×500 ml, 24 hrs), followed by ethyl acetate (3×500 ml, 24 hrs). After evaporation of the latter solvent the residual gum is chromatographed on a Sephadex LH-20 column, eluting with MeOH/CH2C12 (1:1). The fractions containing the TTF, according to a TLC plate, are chromatographed again, twice on Sephadex LH-20 columns and silica gel, using hexane with increasing proportions of ethyl acetate as fluent. TTF is afforded by elution with 40% ethyl acetate in hexane. Infra-red (IR) spectra are obtained with a Bruker Fourier transform infra-red spectra (FTIR) Vector 22 spectrometer. ¹H and ¹³C NMR spectra were recorded on Bruker Avancc-500 spectrometer. Correlation spectroscopy (COSY), heteronuclear single quantum coherence spectroscopy (HSQC) and heteronuclear multiple-bond correlation spectroscopy (HMBC) experiments are recorded using standard Bruker pulse sequences. High resolution electrospray mass spectroscopy (HRESIMS) measurements are performed using the instrument Waters Micromass SYNAPT HDMS mass spectrometer, time of flight (TOF).

TTF may also be expressed in plants by upregulation of its biosynthetic pathway.

In an embodiment of the invention, TTF is synthetically produced using methods known to a person skilled in the art. Such synthetically produced TTF is referred to herein as “synthetic TTF”.

The following is an exemplary scheme for producing synthetic TTF:

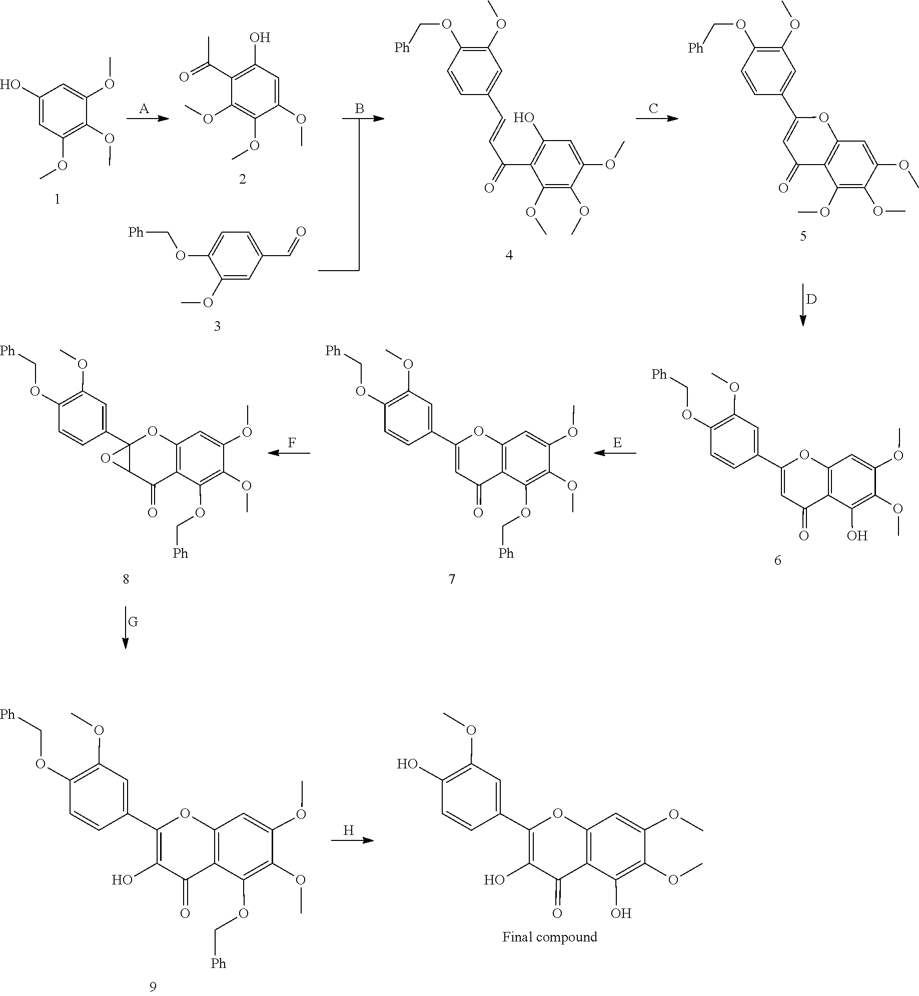

An exemplary procedure for preparing synthetic TTF is shown in the Examples below.

In an embodiment, the TTF according to the present invention is a synthetic TTF produced as described above.

In an embodiment, the present invention provides a method of producing synthetic TTF, the method comprising steps as shown in the Examples below.

In an embodiment of the invention, TTF is administered as a prodrug.

The terms “treating”, “preventing”, “ameliorating” and the like are used herein to generally mean obtaining a desired pharmacological and/or physiological and/or biological effect. The effect may be prophylactic in terms of completely or partially preventing the eye disease or symptom thereof and/or may be therapeutic in terms of partially or completely curing the eye disease and/or an adverse effect attributed to the disease. The term “treatment” as used herein includes any treatment of an eye disease in a mammal, particularly a human, and comprises: (a) preventing the disease from occurring in a subject which may be predisposed to the disease but has not yet been diagnosed as having it; (b) inhibiting the disease, i.e., arresting or slowing its development; (c) relieving the disease, i.e., causing regression of the disease or (d) providing adjunct treatment to various ocular therapeutic procedures. The present invention is directed towards treating patients with medical conditions of the eye.

In certain embodiments, the disease or disorder is selected from retinal degenerative diseases.

In embodiments, the disease or disorder is selected from the group consisting of retinitis pigmentosa (RP), Diabetic retinopathy (DR), chorioretinitis, choroiditis, retinitis, retinochoroiditis, solar retinopathy, choroidal degeneration, choroideremia, hypertensive retinopathy, retinopathy, retinopathy of prematurity, age-related macular degeneration (AMD), macular degeneration, bull's eye maculopathy, epiretinal membrane, peripheral retinal degradation, hereditary retinal dystrophy, retinal hemorrhage, central serous retinopathy, glaucoma, optic neuropathy, Leber's hereditary optic neuropathy, optic disc drusen, skleritis, keratitis, corneal ulcer, arc eye, Thygeson's superficial punctate keratopathy, corneal neovascularization, corneal dystrophy, Fuchs' dystrophy, keratoconus, keratoconjunctivitis sicca, herpes, dry eye, iritis, and uveitis, optic neuritis, bacterial infections (e.g. Lyme disease), viral infections (e.g. measles, mumps), sarcoidosis, lupus neuromyelitis optica, eye complications associated with use of medications (e.g., quinine, antibiotics), optic nerve degeneration, ischemic optic neuropathy (e.g., Non-Arteritic Anterior Ischemic Optic Neuropathy (NAION), Anterior Ischemic Optic Neuropathy (AION), Posterior Ischemic Optic Neuropathy (PAION)).

In specific embodiments, the disease or disorder is selected from RP, AIMD, DR, and optic nerve degeneration.

In one embodiment, said treating comprises an adjunct treatment for an ocular therapeutic procedure.

As used herein, the term “adjunct treatment” or “adjunctive therapy” refers to the use of TTF as a secondary treatment assisting a primary treatment, e.g., a subretinal, intravitreal or suprachoroidal therapeutic delivery surgery.

Therefore, in an embodiment, the ocular therapeutic procedure comprises ocular, subretinal, intravitreal or suprachoroidal therapeutic delivery surgery.

In some embodiments, said subretinal, intravitreal or suprachoroidal therapeutic delivery surgery comprises delivery of gene therapy, stem cell therapy, and/or a prosthesis delivery.

In some embodiments, the subject is a human.

In some other embodiments, the subject is selected from sheep, pigs, cattle, goats, horses, camels, buffalo, rabbits, cats, dogs, and primates.

TTF or the compositions as described in the instant invention can be administered in various routes, such as, without limitation, by topical, cutaneous, subcutaneous, transdermal, conjunctival, subconjunctival, intracorneal, intraocular, ophthalmic, oral, and/or parenteral administration.

As used herein the term conjunctival administration refers to administration to the conjunctiva, the delicate membrane that lines the eyelids and covers the exposed surface of the eyeball.

In some embodiments, TTF or the compositions as described in the instant invention are administered, or adapted for administration, to the eye in the form of an eye drop solution, suspension, cream, ointment, paste, gel, spray, aerosol, foam, a microparticle or a nanoparticle formulation, a solid insert or administered using an ophthalmic device.

TTF or the compositions as described herein are administered to a subject in a therapeutically effective amount. The term “therapeutically effective amount”, in the context of the present disclosure, refers to the amount of the compounds or compositions described herein that will elicit the desired therapeutic effect or response or provide the desired benefit when administered in accordance with the desired treatment regimen. Specifically, such effective amount relates to the amount which is competent for treatment, prevention, or amelioration of eye diseases.

As shown in the Examples below a concentration of 8 nM TTF (see e.g., Example 1) or 12.5 mg/ml TTF (see e.g., Examples 4-7) was positively effective in protecting retinal cone photoreceptors from apoptosis in vitro and in rescuing retinal function in an animal model. A person skilled in the art would realize that such an effective concentration could be adjusted according to the components of the formulation and the mode of administration. For example, if the composition is administered close to the retina, for example by injection or using an implant, the concentration may be in the ng/ml range, while in cases that the composition is administered in the form of eye drops or an implant/injection in locations further away from the retina (e.g., sub-tenon injection) the required concentration is higher and may be in the mg/ml range. Therefore, in some embodiments TTF or the compositions as described in the instant invention is administered at a concentration of between about 0.3 ng/ml and 120 mg/ml, for example, but not limited to, 0.36 ng/ml, 2.5 ng/ml, 3.6 ng/ml, 36 ng/ml, 100 ng/ml, 0.36 mg/ml, 1.25 mg/ml, 4 mg/ml, 12.5 mg/ml or 120 mg/ml.

In some embodiments, TTF or the compositions as described in the instant invention is administered once daily, twice daily, thrice daily or four times a day, wherein each is an embodiment of the invention, depending on the nature of the eye disease and as per the physician's recommendations.

As a non-limiting example, if the patient suffers from a chronic ophthalmic disease TTF or the compositions of the invention may be administered once daily for a predetermined or unlimited amount of time. In other cases, whereby the patient suffers from an acute condition, TTF or the compositions of the invention may be administered twice daily, thrice daily or four times a day or more, according to physician's recommendations.

In some embodiments, the administration is every other day.

In another embodiment, the administration is once weekly. In some other embodiments, the administration is once a month.

In one embodiment, wherein the patient undergoes gene therapy or any other surgery including subretinal, intravitreal or suprachoroidal treatment delivery, TTF or the composition of the invention is administered for 1-4 weeks before and after treatment.

In some other embodiments, for instance when the composition is administered via an ophthalmic device (e.g., a slow-release ophthalmic device that may be refilled), the administration is once a year, once every few months, once a month or once in a few weeks. The composition may also be administered using a gel (e.g., hydrogel, silk gel) or any other chemical formulation suitable for slow release.

When referring to the term “pharmaceutically acceptable”, the general meaning in the context of the disclosure is the suitability of the carrier/material for administration to a mammal, including humans without being toxic or non-safe.

In some embodiments, pharmaceutical/ophthalmic compositions of the invention are administered by dropping, spraying, injecting intraocularly or by release from an ophthalmic device.

In some further embodiments, the invention provides concomitant administration of compounds of the invention together with another active agent.

In some embodiments, the active agent is selected from the group consisting of achilloide A, acetazolamide, acetylcysteine, acyclovir, antazoline, xylometazoline, apraclonidine, atropine, azelastine, azithromycin, betamethasone, betaxolol, bimatoprost, brimonidine, brinzolamide, bromfenac, liquid carbomer, carmellose sodium, carteolol, chloramphenicol, ciprofloxacin, cyclopentanolate, dexamethasone, diclofenac, dorzolamide, emedastine, epinastine, fluorometholone, flurboprofen, fisuduc acid, ganciclovir, gentamicin, homatropine, Hypromellose, ketorolac, ketotifen, latanoprost, levobunolol, levofloxacin, lodoxamide, loteprednol, moxifloxacin, nedocromil sodium, nepafenac, ofloxacin, olopatadine, pilocarpine, polyvinyl alcohol, prednisolone, rimexolone, sodium cromoglicate, sodium hyaluronate, soybean oil, tafluprost, timolol, tobramycin, travoprost, tropicamide, vitamin A, vitamin E, omega 3, vitamin C, beta-carotene, zinc oxide, statins, VEGF inhibitors and inhibitor like drugs.

In embodiments, the VEGF inhibitors and inhibitor-like drugs are selected from ranibizumab, bevacizumab, pegaptanib, aflibercept and brolucizumab.

In some other embodiments, compounds of the invention are given together with a photodynamic therapy, laser therapy, radiation therapy, adjuvant therapy, surgery or stem cells therapy.

In another aspect, the invention provides a pharmaceutical composition for treating, preventing, or ameliorating an eye disease or disorder in a subject, the composition comprising 3,5,4′-trihydroxy-6,7,3′-trimethoxyflavone (TTF) or a pharmaceutically acceptable salt or solvate thereof and one or more pharmaceutically acceptable carriers.

In another aspect, the invention provides an ophthalmic composition comprising TTF or a pharmaceutically acceptable salt or solvate thereof, and an ophthalmic acceptable carrier.

In some embodiments, the ophthalmic composition is in the form of a solution, suspension, ointment, paste, spray, aerosol, foam, microparticle or a nanoparticle formulation, or a gel.

In a specific embodiment, said ophthalmic composition is an eye drop.

The compositions according to the invention may be conveniently admixed with a non-toxic pharmaceutical organic carrier, or with a non-toxic pharmaceutical inorganic carrier. Pharmaceutically acceptable carriers may comprise, for example, water, mixtures of water and water-miscible solvents such as lower alkanols or aralkanols, peanut oil, vegetable oils, polyalkylene glycols, ethyl oleate, ethyl cellulose, petroleum-based jelly, carboxymethyl-cellulose, polyvinylpyrrolidone, isopropyl myristate, saline, polyvinyl alcohols, and other conventionally pharmaceutically acceptable carriers.

The composition may also contain a nontoxic excipient such as emulsifying, preserving, wetting agents, bodying agents and the like, as for example, polyethylene glycols, carbowaxes, antibacterial agents such as quaternary ammonium compounds, phenylmercuric salts, phenyl ethanol, methyl and propyl paraben, benzyl alcohol, thimerosal, buffering agents such as sodium acetates, sodium borates, gluconate buffers, and other conventional agents such as oleate, triethanolamine, thiosorbitol, polyoxyethylene sorbitan monopalmitylate, dioctyl sodium sulfosuccinate, monothioglycerol, sorbitan monolaurate, ethylenediamine tetracetic acid, and the like.

When referring to the term “ophthalmic acceptable carrier”, the general meaning in the context of the disclosure is the suitability of the carrier/material for administration to the eye without being toxic or non-safe.

Suitable ophthalmic acceptable carriers can be used as adequate carriers for the present purpose including phosphate buffer vehicle systems, isotonic boric acid, isotonic sodium chloride, isotonic sodium borate, hydroxyethyl cellulose, methylcellulose, polyvinyl alcohol, and saline.

In some embodiments, the ophthalmic compositions of the invention further comprise one or more of a buffering agent, an isotonizing agent, a solubilizer, a preservative, a viscosity-increasing agent, a chelating agent, an antioxidizing agent, an antibiotic, sugar, a pH regulator.

In some specific embodiments, the pharmaceutical composition/ophthalmic compositions may also be in the form of a microparticle formulation and nanoparticle formulation.

In further specific embodiments, the pharmaceutical composition or the ophthalmic composition may also be administered in a controlled/sustained release form using a chemical compound such as, but not limited to, hydrogels, or silk gels.

Accordingly, in an embodiment, the ophthalmic composition is a sustained release composition.

In further specific embodiments, the pharmaceutical composition or the ophthalmic composition may also be administered in an ophthalmic device.

For example, a solid water-soluble polymer may be used as the carrier for the compounds in the ophthalmic device. Suitable polymers which can be utilized to form the ophthalmic device may be any water soluble nontoxic polymer, for example, cellulose derivatives such as methylcellulose, sodium carboxymethyl cellulose, (hydroxyloweralkyl cellulose), hydroxy ethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; chitosan and chitosan derivatives, polysaccharide-based nanocarriers, the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

In some further embodiments, the ophthalmic device is a biodegradable or a non-biodegradable delivery device.

In embodiments, the device is a controlled/sustained release device. In some other embodiments, the device releases the drug in an immediate manner.

In specific embodiments, the ophthalmic device is in the form selected from the group consisting of a contact lens, punctal plug, scleral patch, scleral ring, Cul-de sac insert, subconjunctival implant, episcleral implant, sub choroidal implant and intravitreal implant.

In some embodiments, the device is a non-invasive drug delivery device such as topical ophthalmic drug delivery device (TODD).

The pharmaceutical preparation may contain nontoxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitanmonopalmitylate, ethylenediamine tetraacetic acid, and the like.

For topical ocular administration, the novel compositions of this invention are formulated so that a unit dosage comprises a therapeutically effective amount of the active component or some multiple thereof in the case of a combination therapy.

As will be shown in the Examples below, the inventors demonstrated that supplementation of TTF at nanomolar concentrations rescued cone photoreceptors from degeneration in vitro in retinal cultures derived from the RPE65/rd12 mouse model of RP.

Moreover, in in-vivo studies performed in the RPE65/rd12 mouse model of RP, daily treatment (twice a day) with eye drops containing TTF resulted in the following significant effects:
(i) microglial activation in the retina was reduced,
(ii) IL-1β concentration in the retina was reduced,
(iii) photoreceptor cell death was prevented,
(iv) the treatment rescued retinal function in vivo as demonstrated by a significantly higher mean maximal dark-adapted electroretinogram (ERG) a-wave and b-wave results in TTF treated mice vs. control mice treated with the vehicle alone.
(v) no side effects were observed (as indicated by observing the animals' general health, as well as the retinal and corneal structure) following 12 weeks of daily treatment with the TTF eye drops.

Without wishing to be bound by theory, the positive effect of TTF on rescuing photoreceptors from degeneration and its positive effect on retinal function may be mediated by TTF activity of reducing oxidative stress in the retinal neurons and inhibiting signaling pathways that mediate photoreceptors cell death. In addition, the effect of TTF may also be mediated at least partly by inhibition or reduction of immune activity in the retina.

Diseases related to the degeneration of retinal cells, are often associated with activation of retinal immune cells.

In most cases, retinal immune cells which participate in the inflammatory processes of the eye, especially those involved in degradation of retinal cells and photoreceptors are microglia, macroglia and mononuclear monocytes.

Thus, in yet another aspect, the present invention provides TTF, a pharmaceutically acceptable salt or solvate thereof, or an ophthalmic composition comprising same for use in inhibition or reduction of retinal immune cells activation.

In some embodiments, the retinal immune cells are selected from microglia, macroglia and mononuclear monocytes.

In yet another aspect, the present invention provides TTF, a pharmaceutically acceptable salt or solvate thereof, or an ophthalmic composition comprising same for use in reducing the levels of cytokines in the eye. In some embodiments, the cytokines are selected from IL-6, IL-1β,IL-2, IL-4, IL-6, IL-8, IL-10, IFN-γ, GRO-α and I-309. In some specific embodiments, the cytokines are IL-6 and/or IL-1β.

In yet another aspect, the present invention provides TTF, a pharmaceutically acceptable salt or solvate thereof, or an ophthalmic composition comprising same for use in inhibiting or reducing the death of photoreceptors and for inhibiting, reducing, or preventing retinal cells degeneration.

One of the hallmarks of various eye pathologies is the presence of extracellular deposits of lipids and proteins called drusen, primarily in the central region of the retina.

Therefore, in yet another aspect, the present invention provides TTF, a pharmaceutically acceptable salt or solvate thereof, or an ophthalmic composition comprising same for use in decomposing or disassembling drusen.

In a further aspect, the invention provides a compound having the structural formula I.

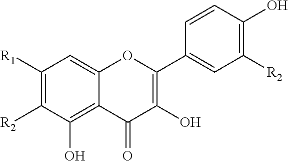

wherein R1, R2 and R3 are each independently selected from —H, —(C1-C4)hydroxyalkyl, —C(═O)H, —C(═O)—OH, —C(═O)—(C1-C4)alkyl, —C(═O)—(C1-C4)alkenyl, —C(═O)—(C1-C4)alkynyl, —(C1-C4)alkyl, —(C1-C4)alkenyl, —(C1-C4)alkynyl, —(C1-C4)haloalkyl, —(Cl-C4)alkoxy, wherein at least one of R1, R2 and R3 is different from —H, for use in treating, preventing or ameliorating an eye disease or disorder.

In some embodiments, R1, R2 and R3 are each independently selected from —(C1-C4)hydroxyalkyl, —(C1-C4)alkyl, —(C1-C4)alkenyl, —(C1-C4)alkynyl and —(C1-C4)alkoxy.

In some embodiments, R1, R2, and R3 are each independently —(C1-C4)alkoxy.

In other embodiments there is provided a compound, having the structural formula II.

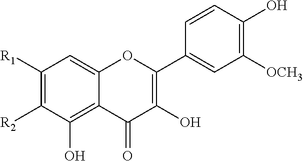

wherein, R1 and R2 are each independently selected from —H, —(C1-C4)hydroxyalkyl, —C(═O)H, —C(═O)—OH, —C(═O)—(C1-C4)alkyl, —C(═O)—(C1-C4)alkenyl, —C(═O)—(C1—C4)alkynyl, —(C1-C4)alkyl, —(C1-C4)alkenyl, —(C1-C4)alkynyl, —(C1-C4)haloalkyl, —(Cl—C4)alkoxy, wherein at least one of R1 and R2 is different from —H. The compound is for use in treating, preventing, or ameliorating an eye disease or disorder.

In some other embodiments, the compound is as described herein, wherein R1 and R2 are each independently selected from —(C1-C4)hydroxyalkyl, —(C1-C4)alkyl, —(C1—C4)alkenyl, —(C1-C4)alkynyl and —(C1-C4)alkoxy.

In other embodiments, R1 and R2 are each independently —(C1-C4)alkoxy.

In yet further embodiments the invention provides a compound, having the structural formula III:

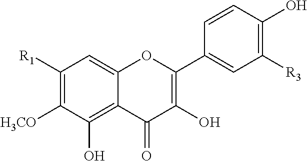

wherein, R1 and R3 are each independently selected from —H, —(C1-C4)hydroxyalkyl, —C(═O)H, —C(═O)—OH, —C(═O)—(C1-C4)alkyl, —C(═O)—(C1-C4)alkenyl, —C(═O)—(C1—C4)alkynyl, —(C1-C4)alkyl, —(C1-C4)alkenyl, —(C1-C4)alkynyl, —(C1-C4)haloalkyl, —(Cl—C4)alkoxy, wherein at least one of R1 and R3 is different from —H. The compound is for use in treating, preventing, or ameliorating an eye disease or disorder.

In embodiments, the compound is as described herein, wherein R1 and R3 are each independently selected from —(C1-C4)hydroxyalkyl, —(C1-C4)alkyl, —(C1—C4)alkenyl, —(C1-C4)alkynyl and —(C1-C4)alkoxy.

In other embodiments, R1 and R3 are each independently —(C1-C4)alkoxy.

In yet further embodiments, the invention provides compounds, having the structural formula IV:

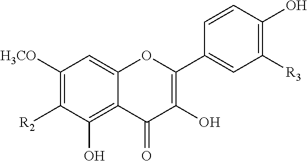

wherein, R2 and R3 are each independently selected from —H, —(C1-C4)hydroxyalkyl, C(═O)H, —C(═O)—OH, —C(═O)—(C1-C4)alkyl, —C(═O)—(C1-C4)alkenyl, —C(═O)—(C1—C4)alkynyl, —(C1-C4)alkyl, —(C1-C4)alkenyl, —(C1-C4)alkynyl, —(C1-C4)haloalkyl, —(Cl—C4)alkoxy, wherein at least one of R2 and R3 is different from —H, and wherein the compound is for use in treating, preventing or ameliorating an eye disease or disorder.

In some embodiments, R2 and R3 are each independently selected from —(C1—C4)hydroxyalkyl, —(C1-C4)alkyl, —(C1-C4)alkenyl, —(C1-C4)alkynyl and —(C1—C4)alkoxy.

In yet other embodiments, R2 and R3 are each independently —(C1-C4)alkoxy.

In yet further embodiments, the invention further provides compounds, having the structural formula V:

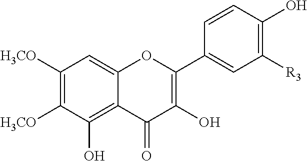

wherein, R3 is selected from —H,—(C1-C4)hydroxyalkyl, —C(═O)H, —C(═O)—OH, —C(═O)—(C1-C4)alkyl, —C(═O)—(C1-C4)alkenyl, —C(═O)—(C1-C4)alkynyl, —(C1-C4)alkyl, —(Cl—C4)alkenyl, —(C1-C4)alkynyl, —(C1-C4)haloalkyl, —(C1-C4)alkoxy, wherein the compound is for use in treating, preventing or ameliorating an eye disease or disorder.

In some embodiments, R3 is selected from —(C1-C4)hydroxyalkyl, —(C1-C4)alkyl, —(C1-C4)alkenyl, —(C1-C4)alkynyl and —(C1-C4)alkoxy.

In some embodiments, R3 is —(C1-C4)alkoxy.

In supplementary embodiments, the invention provides compounds having the structural formula VI:

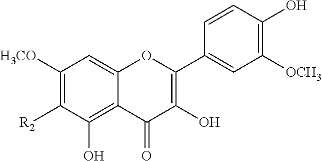

wherein, R2 is selected from —H, —(C1-C4)hydroxyalkyl, —C(═O)H, —C(═O)—OH, —C(═O)— ^{PGP}-2⁸,C1 (C1-C4)alkyl, —C(═O)—(C1-C4)alkenyl, —C(═O)—(C1-C4)alkynyl, —(C1-C4)alkyl, —(Cl—C4)alkenyl, —(C1-C4)alkynyl, (C1-C4)haloalkyl, —(C1-C4)alkoxy, wherein the compound is for use in treating, preventing or ameliorating an eye disease or disorder.

In further supplementary embodiments, R2 is selected from —(Cl—C4)hydroxyalkyl, —(C1-C4)alkyl, —(C1-C4)alkenyl, —(C1-C4)alkynyl and —(C1—C4)alkoxy.

In embodiments, R2 is —(C1-C4)alkoxy.

In certain embodiments, the invention stipulates a compound having the structural formula VII:

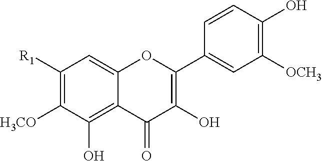

wherein, R1 is selected from —(C1-C4)hydroxyalkyl, —C(═O)H, —C(═O)—OH, —C(═O)—(C1—C4)alkyl, —C(═O)—(C1-C4)alkenyl, —C(═O)—(C1-C4)alkynyl, —(C1-C4)alkyl, —(Cl—C4)alkenyl, —(C1-C4)alkynyl, —(C1-C4)haloalkyl, —(C1-C4)alkoxy, for use in treating, preventing or ameliorating an eye disease or disorder.

In embodiments, R1 is selected from —(C1-C4)hydroxyalkyl, —(C1-C4)alkyl, —(C1—C4)alkenyl, —(C1-C4)alkynyl and —(C1-C4)alkoxy.

In other embodiments, R1 is —(C1-C4)alkoxy.

The following examples are representative of techniques employed by the inventors in carrying out aspects of the present invention. It should be appreciated that while these techniques are exemplary embodiments for the practice of the invention, those of skill in the art, in view of the present disclosure, will recognize that numerous modifications can be made without departing from the spirit and intended scope of the invention.

### EXAMPLES

### Experimental Procedures

Preparation of Synthetic TTF

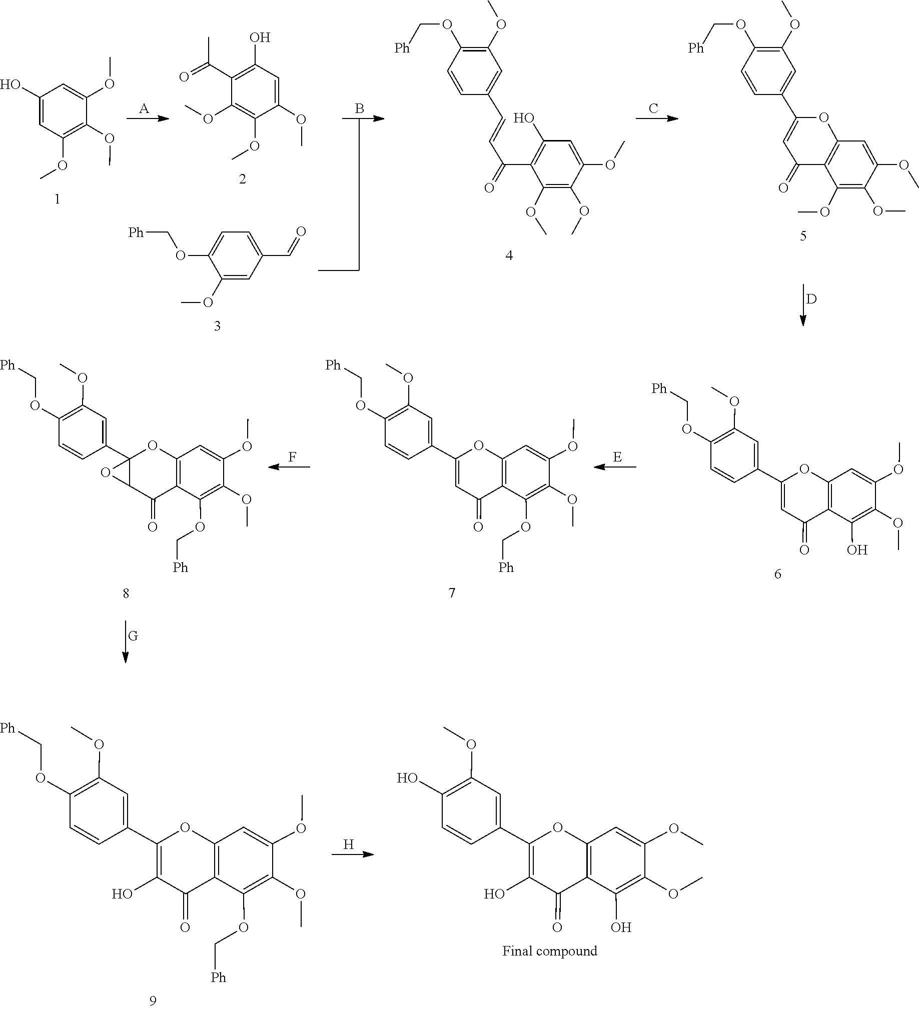

### Step A:

BF₃·Et₂O (6 mL, 48.6 mmol) was added dropwise to a solution of compound 1 (3.00 g, 13.3 mmol) in glacial acetic acid (10 mL). The reaction mixture was stirred at 70° C. until TLC analysis (30% ethyl acetate/pet ether) showed completion of reaction (approx. for 2 h). Then the reaction mixture was quenched with water and filtered to give 1.90 g (8.40 mmol, 48%) of compound 2 pure enough for the next step, as a yellow solid.

### Step B:

To a cooled to 0° C. stirred solution of compound 2 (20.0 g, 88.4 mmol) and aldehyde 3 (17.8 g, 73.8 mmol) in ethanol (800-1000 mL) freshly powdered KOH (19.3 g, 344 mmol) was added. The mixture was slowly allowed to warm to room temperature and stirred for 96 h. The solvent was evaporated to approximately one-fifth of the original volume. Ice-cold water (˜2 mL) was added, and the mixture was neutralized with 2N hydrochloric acid. Then, MTBE (600 mL) was added, and the mixture was filtered. The precipitate was recrystallized from ethyl acetate/MTBE mixture (300/300 mL) to yield 18.0 g (40.0 mmol, 54%) of compound 4 as an orange solid.

### Step C:

To a cooled to 0° C. stirred solution of compound 4 (0.450 g, 0.999 mmol) and PIDA (0.390 g, 1.20 mmol) in ethanol (50 mL) freshly powdered KOH (0.170 g, 3.03 mmol) was added. The mixture was slowly allowed to warm up to r.t. and stirred for 96 h. The solvent was evaporated, and the mixture was neutralized with 2N hydrochloric acid. MTBE (20 mL) was added, and the obtained suspension was filtered. The resulting solid was recrystallized from ethyl acetate/MTBE mixture (1/1 mL) to yield 0.200 g (0.446 mmol, 45%) of compound 5 as a yellow solid.

### Step D:

To an ice-cooled stirred solution compound 5 (5.00 g, 11.1 mmol) in acetonitrile (50 mL) AlCl₃ (25.0 g, 187 mmol) was added in small portions and the reaction mixture was stirred overnight at room temperature. The solvent was evaporated, and the mixture was neutralized with 2% aqueous hydrochloric acid. The product was extracted with dichloromethane (100 mL). The organic phase was separated, dried over Na₂SO₄, and evaporated to give 3.20 g (7.37 mmol, 60%) of compound 6, sufficiently pure for the next step.

### Step E:

To a solution of compound 6 (3.20 g, 7.37 mmol) in CH3CN (150 mL) K₂CO3 (3.00 g, 22.2 mmol) and benzyl bromide (175 mL, 14.7 mmol) were added, and the reaction mixture was refluxed overnight. The solvent was evaporated, and the crude product was charged on silica gel column. Elution of the column with 50% ethyl acetate/petroleum ether gave 1.20 g (2.29 mmol, 31%) of compound 7.

### Step F:

To an ice-cooled stirred solution compound 7 (0.500 g, 0.953 mmol), NaHCO₃ (1.00 g, 11.9 mmol) and Na₂CO₃ (2g) in acetone/DCM/H20 mixture (15 mL/20 mL/10 mL) a solution of oxone (7.50 g, 49.3 mmol) in water (30 mL) was slowly added and the reaction mixture was stirred overnight at rt. Then water (100 mL) and dichloromethane (100 mL) were added. The organic phase was separated, dried over Na₂SO₄, and evaporated under reduced pressure to give 0.500 g of crude compound 8.

### Step G:

To a solution of compound 8, obtained in the previous step, in C-1₃CN (50 mL) catalytic amount of PTSA hydrate was added and the reaction mass was stirred at r.t. overnight. The solvent was evaporated, and the crude product was charged on silica gel column. Elution of the column with 40% ethyl acetate/petroleum ether gave 0.100 g (0.185 mmol, 19% over 2 steps) of compound 9.

### Step H:

A mixture of compound 9 (5.00 g, 9.26 mmol) and a catalytic amount of Pd/C (10% wt.) in methanol/ethyl acetate 1:1 mixture (400 mL) was stirred under hydrogen atmosphere at room temperature for 24 h. The catalyst was filtered off and the filtrate was evaporated under reduced pressure. The crude product was recrystallized from methanol to give 2.00 g (5.55 mmol, 60%) of the final compound as a sticky yellowish solid.

### Preparation of Eye Cup Cultures

Eyes were obtained from 21-day old RPE65/rd12 mice, and the posterior segment was separated from the cornea, the lens, the iris, and the ciliary body under a surgical microscope. The eye cups were placed onto a microporous membrane (30 mm in diameter; Millicell-CM; Millipore, Bedford, MA) with the ganglion cell layer (GCL) facing up and the sclera facing the filter in a six-well culture plate. Each well contained 1 ml of culture medium consisting of 50% minimum essential medium/HEPES (Sigma, St. Louis, MO), 25% HBSS (Invitrogen, USA), and 25% heat inactivated fetal bovine serum (Invitrogen, USA) supplemented with 200 μM L-glutamine and 5.75 mg/ml glucose and 8 nM natural TTF (nTTF) or synthetic TTF (sTTF) or same volume of vehicle solution (0.0115% DMSO, control). The eye cups were maintained in culture at 37° C., 5% C02 incubator for 18 hours.

### Preparation of Tissue Sections

The eye cups were fixed with 4% paraformaldehyde (PFA) and embedded in sucrose for cryopreservation. Eight micrometer sections along the vertical meridian of the eye through the optic nerve were cut using a cryostat.

Staining with Iba-1 Antibodies

Eyes were lightly fixed in 4% PFA. The cornea, iris and lens were removed, the neuro-retina was peeled off and the RPE flat mount was incubated with Iba-1 antibodies (Wako chemicals, USA, 1:200 in PBS and 0.1% triton-X) for 16 hours at 4° C., followed by incubation with secondary Alexa Fluor® 488-AffiniPure Donkey Anti-Mouse IgG antibody (Jackson Immuno Research; USA, 1:400 in PBS with 0.2% DAPI) for 2 hours at room temperature. Samples were visualized using a fluorescent microscope (Olympus BX51) and recorded using the Olympus DP71 camera.

### Example 1

TTF Protects Retinal Cone Photoreceptors from Apoptosis In-Vitro:

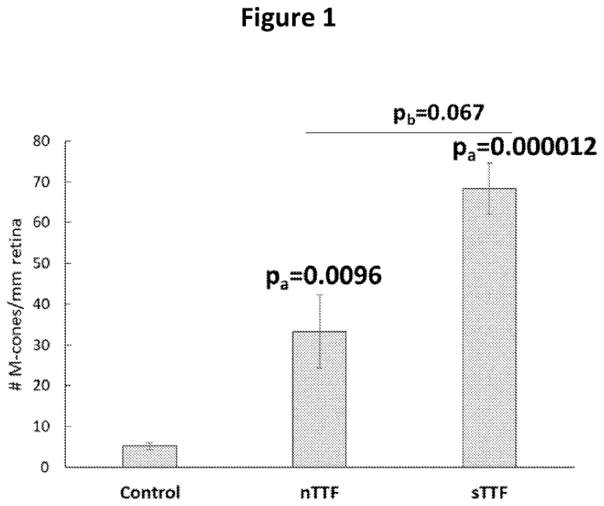

The present example demonstrates that supplementing the growth media with TTF attenuates cone photoreceptor cell death in cultures of retinas from RPE65/rd12 mice cultured in vitro (FIG. 1).

Cultures were prepared from eye cups of 21-day old RPE65/rd12 mice as described in Experimental Procedures above and were incubated in media supplemented with 8 nM natural TTF (nTTF) or synthetic TTF (sTTF) or same volume of vehicle solution (0.0115% DMSO, control) for 18 hours. nTTF was produced as described in WO 2015/079390.

sTTF was synthesized using the following process:

Sections from these eye cups were stained with antibodies directed against M-cone opsin (Milipore, USA) by incubating the sections with antibodies diluted 1:100 in 1% BSA (Sigma) for 16 hrs at 4° C. followed by extensive washing in PBS and 1 hour incubation with 488-AffiniPure Donkey Anti-Mouse IgG antibody (Jackson Immuno Research) at room temperature. Sections were counter-stained with DAPI (Bar-Naor, Israel). The number of positively stained cells/mm retina was recorded. Data are presented as mean±SE.

### Example 2

TTF Crosses the Corneal Barrier into the Anterior Segment

The present example demonstrates that TTF can cross the corneal barrier and penetrate the eye. TTF eye drops (12.5 mg/ml=34.7 mM in DMSO) or vehicle (DMSO) were dropped every 10 seconds on rabbit eyes (10 eye drops of 50 μL per eye−0.5 mL total volume). The eye fluids were collected 45 minutes or 3 hours later. About 100 microliters of anterior chamber fluids were collected from each eye. The eye fluids from both eyes of the same rabbit were pooled and the amount of TTF that penetrated the cornea was determined by IPLC analysis. Twenty microliters of pooled eye fluids were filtered and injected into an Agilent UIHPLC Infinity II 1290 device equipped with a Kinetex 5 μm EVO C18 column (250*4.6 mm). Running conditions were as follows: a 10-minute gradient was delivered at a flow rate of 1 mL/min: 5 minutes in a binary mobile phase which consisted of 0.5% acetic acid (in distilled water), and 5 minutes in acetonitrile in DDW. The detection was performed at a wavelength of 350 nm since, in eye fluids taken from naive rabbits or rabbits given eye drops containing DMSO only, there was no background at 350 nm interfering with the measurement. Under these conditions, the retention time of TTF is 5.086 minutes.

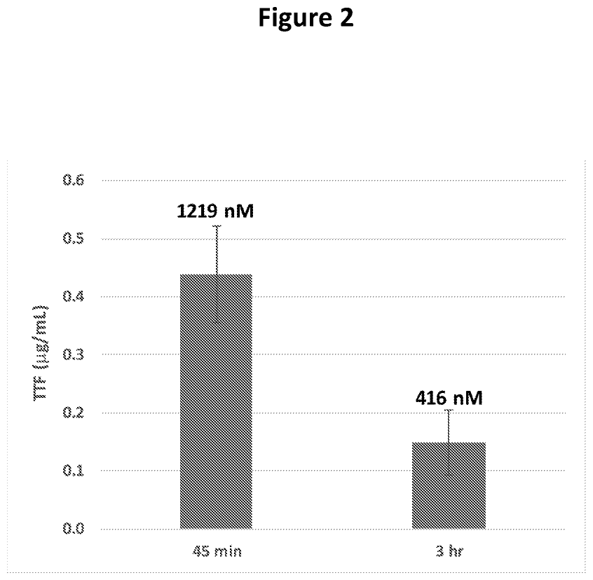

As shown in FIG. 2, a maximal concentration of TTF was demonstrated 45 minutes following eye drops instillation.

### Example 3

### Microglial Activation During Retinal Degeneration in the RPE65/Rd12 RP Mouse Model

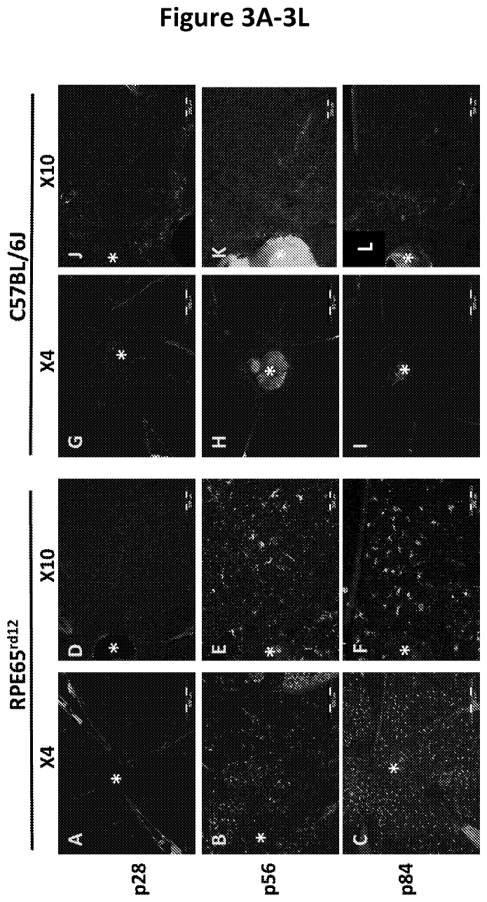

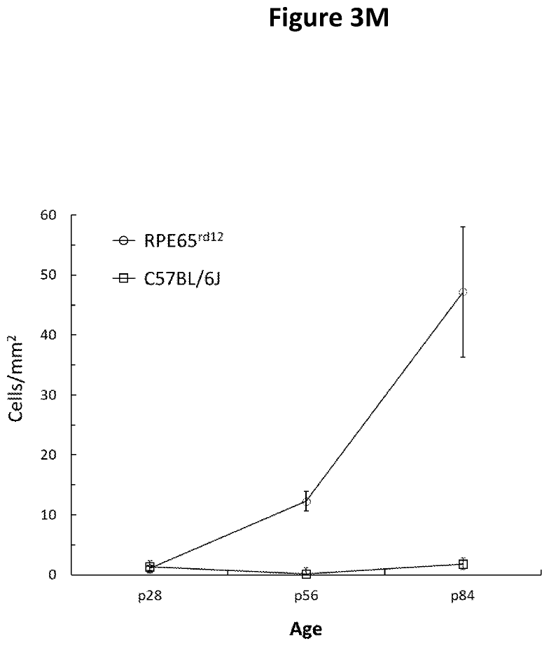

The present example shows an association between microglial cell activation and photoreceptor degeneration in RPE65/rd12 mice, a model for retinitis pigmentosa (RP) due to retinoid cycle defect. Retina flat mounts of RPE65/rd12 mice and wild type C57BL mice were prepared as described above and stained for the microglial specific marker Iba-1. As can be seen in FIG. 3 activation and infiltration of microglial cells into the sub-retina can be seen in retina of RPE65/rd12 mice in contrast with retina of C57BL mice.

### Example 4

### TTF Eye Drops Treatment Prevents Activation and Migration of Microglial Cells in RPE65/Rd12 RP Mouse Model

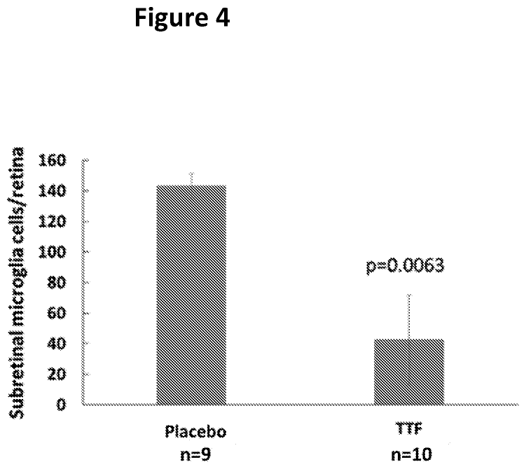

3 weeks old RPE65/rd12 mice were treated twice daily (6 days/week) for 3 weeks with eye drops containing 12.5 mg/ml (34.7 mM) TTF in DMSO (10pl/eye drop) or vehicle alone (DMSO). Each treatment consisted of one eye drop. Retina flat mounts of the treated and the control RPE65/rd12 mice were prepared as described above and stained for the microglial specific marker Iba-1. As shown in FIG. 4, TTF treatment (n=10 mice) significantly reduced microglial activation and migration into the sub retina compared to placebo treatment (n=9 mice, mean±SD: 42±14 microglia cells/retina vs. 143±28 microglia cells/retina, p=0.0063).

### Example 5

### TTF Eye Drops Treatment Reduces IL-1p Concentration in the Retina of RPE65/Rd12 RP Mouse Model

3 weeks old RPE65/rd12 mice were treated twice daily (6 days/week) for 3 weeks with eye drops containing 12.5 mg/ml (34.7 mM) TTF in DMSO (10p1/eye drop) or vehicle alone (DMSO).

Each pair of retinas from the same mouse was homogenized in the same tube in 120 μl lysis buffer containing 10 mM TRIS pH 7.5, 100 mM NaCl, 1 mM EDTA, and 0.01% TRITON X-100. Protein concentration in each sample was determined in duplicates using a commercial kit: Pierce™ BCA Protein Assay Kit (23227), Thermo Fisher Scientific, IL, USA. IL-1β levels were tested using a commercial sandwich ELISA kit: Mouse IL-1 beta/IL-1F2 DuoSet ELISA (DY401). R&D Systems, Inc., MN, USA.

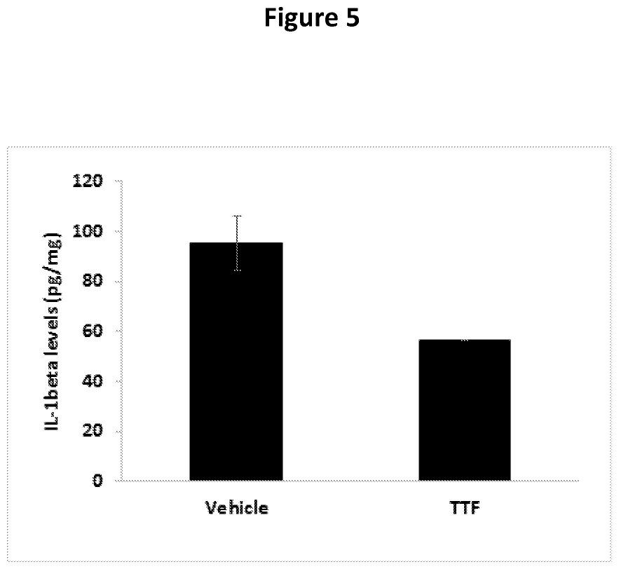

As shown in FIG. 5, TTF treatment reduced IL-1(3 concentration in the retina, suggesting a lower level of inflammation.

### Example 6

TTF Eye Drops Treatment Protects Retinal Photoreceptors from Apoptosis in the RPE65/Rd12 RP Mouse Model

3 weeks old RPE65/rd12 mice were treated twice daily (6 days/week) for 3 weeks with eye drops containing 12.5 mg/ml (34.7 mM) TTF in DMSO (10 μl/eye drop) or vehicle alone (DMSO).

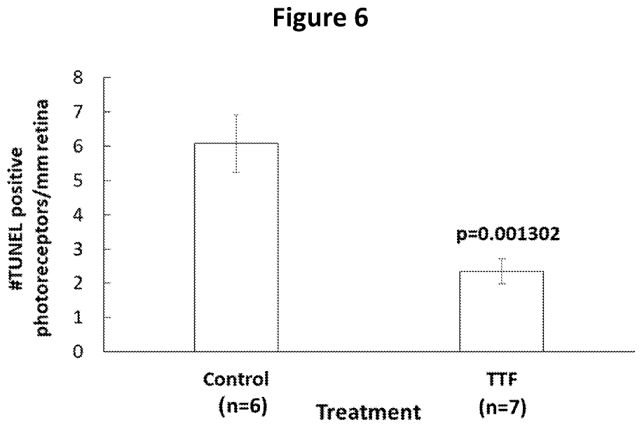

Mice were euthanized using C02, the eyes were obtained and fixed in 10% formalin. Frozen sections were stained with TUNEL-TMR kit following manufacturer's instructions. The number of TUNEL positive nuclei in the outer nuclear layer was counted as well as the length of retinal sections. Results are shown in FIG. 6 as the number of TUNEL positive nuclei/mm retina.

As shown in FIG. 6, TTF treatment protected photoreceptors from apoptosis. Thus, the number of TUNEL positive cells in the photoreceptor layer was significantly lower in mice that received TTF eye drops (n=7) compared with control mice (n=6) that were treated with placebo (mean±SE: 2±0.4 apoptotic cells/mm retina vs. 6±0.8 apoptotic cells/mm, p=0.0013).

### Example 7

### TTF Eye Drops Treatment Preserves Retinal Function in RPE65/Rd12 RP Mouse Model

3 weeks old RPE65/rd12 mice were treated twice daily (6 days/week) for 12 weeks with eye drops (10p1/eye drop) containing 12.5 mg/ml (34.7 mM) TTF (n=6) or vehicle alone (DMSO, n=6).

For dark adapted ERG, the mice were placed in total darkness for at least 12 h prior to examination. Animals were anesthetized with intraperitoneal injection containing 75 mg/kg ketamine and 10 mg/kg xylazine. Pupils were dilated with topical 1% tropicamide and 10% phenylephrine HCl. ERGs were recorded from both eyes simultaneously, using golden wire loop electrodes positioned on each cornea. Chloride silver electrodes were inserted subcutaneously near the temporal canthi as reference. An additional ground electrode was placed on the tail. Tests were performed under dark adapted (scotopic), and light adapted (photopic) conditions. Five light stimuli at increasing intensities were used (0.0023, 0.25, 2.4, 4.4, 23.5 cd-s/m2). For dark adapted ERG, responses were averaged with stimulus intervals of 1 to 30 s depending on the stimulus light intensity.

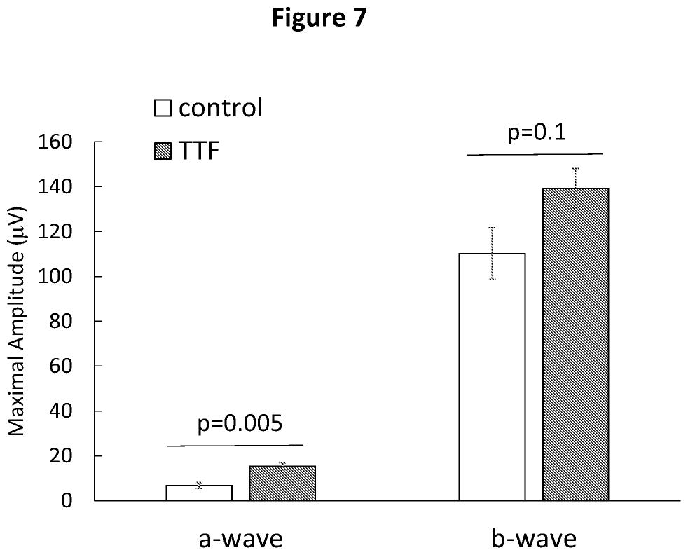

As shown in FIG. 7, TTF treatment preserved photoreceptor function with significantly higher maximal dark-adapted b-wave amplitudes as well as a-wave amplitudes recorded in mice that received TTF eye drops treatment compared with mice that received placebo eye drops, indicating better function of retinal photoreceptors and bipolar cells following TTF eye drop treatment.

1-49. (canceled)

50. An ophthalmic composition comprising 3,5,4′-trihydroxy-6,7,3′-trimethoxyflavone (TTF) or a pharmaceutically acceptable salt or solvate thereof, and an ophthalmic acceptable carrier.

51. The ophthalmic composition according to claim 50, wherein said ophthalmic composition is an eye drop.

52. The ophthalmic composition according to claim 50, wherein the ophthalmic composition is in the form of a solution, suspension, ointment, paste, spray, aerosol, foam, microparticle or a nanoparticle formulation, or a gel.

53. The ophthalmic composition of claim 50, wherein the ophthalmic composition comprises one or more of a buffering agent, an isotonizing agent, a solubilizer, a preservative, a viscosity-increasing agent, a chelating agent, an antioxidizing agent, an antibiotic, a sugar, or a pH regulator.

54. The ophthalmic composition according to claim 50, wherein the ophthalmic acceptable carrier is selected from the group consisting of phosphate buffer vehicle systems, isotonic boric acid, isotonic sodium chloride, isotonic sodium borate, hydroxyethyl cellulose, methylcellulose, polyvinyl alcohol, and saline.

55. The ophthalmic composition according to claim 50, wherein the ophthalmic composition is comprised within an ophthalmic device.

56. The ophthalmic composition according to claim 55, wherein the ophthalmic device is in the form selected from the group consisting of a contact lens, a punctal plug, a scleral patch, a scleral ring, a Cul-de sac insert, a subconjunctival/episcleral implant, a subchoroidal implant, an intravitreal implant, and a non-invasive delivery device such as, a topical ophthalmic drug delivery device (TODD).

57. The ophthalmic composition according to claim 50, wherein the ophthalmic composition is a sustained release composition.

58. The ophthalmic composition according to claim 50, wherein the TTF is isolated from a plant (such as achilleafragranissima) or is a synthetically produced TTF.

59. A method of treating an eye disease or disorder, or a method for providing adjunct treatment to an ocular therapeutic procedure the method comprising administering to a subject in need thereof a therapeutically effective amount of TTF or a pharmaceutically acceptable salt or solvate thereof.

60. The method according to claim 59, wherein the disease or disorder is selected from the group consisting of retinitis pigmentosa (RP), Diabetic retinopathy (DR), chorioretinitis, choroiditis, retinitis, retinochoroiditis, solar retinopathy, choroidal degeneration, choroideremia, hypertensive retinopathy, retinopathy, retinopathy of prematurity, age-related macular degeneration (AMD), macular degeneration, bull's eye maculopathy, epiretinal membrane, peripheral retinal degradation, hereditary retinal dystrophy, retinal haemorrhage, central serous retinopathy, glaucoma, optic neuropathy, leber's hereditary optic neuropathy, optic disc drusen, skleritis, keratitis, corneal ulcer, arc eye, thygeson's superficial punctate keratopathy, corneal neovascularization, corneal dystrophy, fuchs' dystrophy, keratoconus, keratoconjunctivitis sicca, herpes, dry eye, iritis, and uveitis, optic neuritis, bacterial infections (e.g. Lyme disease), viral infections (e.g. measles, mumps), sarcoidosis, lupus neuromyelitis optica, eye complications associated with use of medications (e.g., quinine, antibiotics), optic nerve degeneration, ischemic optic neuropathy (e.g., Non-Arteritic Anterior Ischemic Optic Neuropathy (NAION), Anterior Ischemic Optic Neuropathy (AION), Posterior Ischemic Optic Neuropathy (PAION)).

61. The method according to claim 59, wherein the ocular therapeutic procedure comprises ocular, subretinal, intravitreal or suprachoroidal delivery.

62. The method according to claim 61, wherein the ocular, subretinal, intravitreal or suprachoroidal delivery comprises delivery of gene therapy, stem cell therapy, or a prosthesis.

63. The method according to claim 59, wherein the disease or disorder is selected from AMD, DR, RP, and optic nerve degeneration.

64. The method according to claim 59, wherein said subject is a human.

65. The method according to claim 59, wherein said subject is a mammal selected from the group consisting of sheep, pigs, cattle, goats, horses, camels, buffalo, rabbits, cats, dogs, and primates.

66. The method according to claim 59, wherein said TTF is administered as an eye drop solution, a suspension, an ointment, a paste, a spray, an aerosol, a foam, a microparticle or a nanoparticle formulation, a gel or using an ophthalmic device.

67. The method according to claim 59, wherein said composition is administered at a concentration of between about 0.3 ng/ml and 120 mg/ml.

68. The method according to claim 59, wherein said TTF is administered once, twice, or three times daily.

69. The method of claim 59, wherein the TTF is isolated from a plant (such as Achillea fragranissima) or is a synthetically produced TTF.
